(19) United States
(12) Patent Application Publication
(10) Pub. No.: US 2025/0134420 A1
(43) Pub. Date: May 1, 2025

(54) SYSTEM AND METHOD FOR MANAGING FAT TREND OF A SUBJECT

(71) Applicant: OBCTOR LTD., Hofit (IL)

(72) Inventor: Nilio ECKSTEIN, Hofit (IL)

(73) Assignee: OBCTOR LTD., Hofit (IL)

(21) Appl. No.: 19/010,107

(22) Filed: Jan. 5, 2025

Related U.S. Application Data
(63) Continuation-in-part of application No. PCT/IL2023/050692, filed on Jul. 5, 2023.
(60) Provisional application No. 63/358,342, filed on Jul. 5, 2022.

(51) Int. Cl. A61B 5/145; A61B 5/00

(52) U.S. Cl. CPC A61B 5/14546; A61B 5/1451; A61B 5/742

(57) ABSTRACT

A method and a system for managing fat trend of a subject are disclosed. The system comprises: a sensing unit; and a computing device herein the sensing unit comprises: a first sensor configured to measure a first temporal biomarker value in a body fluid of the subject; a second sensor configured to measure a second temporal biomarker value in the body fluid of the subject; and a communication module configured to transmit the measured temporal biomarkers values to the computing device. The computing device is configured to execute the following method steps: receiving the first temporal biomarker value and the second temporal biomarker value from the sensing unit; calculating a temporal fatness index based on the first temporal biomarker value and the second temporal biomarker value; and displaying the temporal fatness index on a user device.

### CROSS REFERENCE TO RELATED APPLICATIONS

This application is a continuation-in-part of PCT Patent Application No. PCT/IL2023/050692, filed on 5 Jul. 2023, which claims the benefit of priority under 35 U.S.C. § 119 (e) of U.S. Provisional Patent Application No. 63/358,342, filed Jul. 5, 2022. The content of the above applications are all incorporated by reference as if fully set forth herein in their entirety.

### FIELD OF THE INVENTION

The present invention relates generally to methods for combating obesity. More specifically, the present invention relates to a method of combating obesity by managing the fat gain/loss trend of a subject.

### BACKGROUND OF THE INVENTION

Obesity and overweight are major causes of disability and are correlated with various diseases and conditions, particularly cardiovascular diseases, type 2 diabetes, obstructive sleep apnea, certain types of cancer, and osteoarthritis. Obesity is a condition in which excess body fat has accumulated to such an extent that it may have a negative effect on health. High BMI is a marker of risk for, but not a direct cause of, diseases caused by diet and physical activity. As each human is different, different diets and physical activities will have different effects on different humans.

Currently, there is no direct way to measure the tendency of a person to gain or lose fat following a meal or a physical activity.

Lipogenesis (fat gain), the process of fat accumulation, involves the conversion of free fatty acids (FFA) and glycerol into triglycerides, which are then stored as fat within adipocytes (fat cells). It is a metabolic pathway responsible for the synthesis and storage of fats in the body. Insulin, a pivotal hormone in regulating overall metabolism, holds significant influence over fat metabolism, including the process of lipogenesis. Specifically, insulin promotes lipogenesis by stimulating glucose uptake into adipose cells and subsequently enhancing the activities of lipogenic enzymes within them.

Lipolysis, the metabolic process responsible for fat loss, operates as an inverse pathway to lipogenesis. It entails the hydrolysis of triglycerides into glycerol and FFA. Insulin, once again, assumes a critical role as the primary regulatory hormone in controlling lipolysis. This process is triggered when insulin secretion is suppressed and blood insulin levels are low, as commonly observed during fasting. Precise regulation of lipogenesis and lipolysis is essential for maintaining a delicate equilibrium between fat gain and fat loss in the human body, effectively preventing obesity and overweight conditions.

The circadian rhythm refers to the 24-hour internal clock that regulates various physiological processes in living organisms, including humans. This internal clock is primarily influenced by external cues, such as light and darkness, and helps regulate sleep-wake cycles, hormone production, metabolism, and other bodily functions.

Recent research has suggested that aligning one's eating patterns with one's circadian rhythm may have potential health benefits. The concept of “time-restricted feeding” or “intermittent fasting” is one approach that takes the circadian rhythm into account. This involves restricting the eating window to a specific period during the day, typically around 8-12 hours, and fasting for the remaining hours.

Accordingly, there is a need for a device and method that can monitor lipogenesis and lipolysis in the human body in real-time and provide reliable information that may allow a user to combat obesity and overweight. The device may further help to align the eating time with the user's circadian rhythm.

### SUMMARY OF THE INVENTION

Some aspects of the invention are directed to a method and a system for managing fat trend of a subject, the system comprising: a sensing unit; and a computing device herein the sensing unit comprises: a first sensor configured to measure a first temporal biomarker value in a body fluid of the subject; a second sensor configured to measure a second temporal biomarker value in the body fluid of the subject; and a communication module configured to transmit the measured temporal biomarkers values to the computing device. In some embodiments, the computing device is configured to execute the following method steps: receiving the first temporal biomarker value and the second temporal biomarker value from the sensing unit; calculating a temporal fatness index based on the first temporal biomarker value and the second temporal biomarker value; and displaying the temporal fatness index on a user device.

In some embodiments, calculating the temporal fatness index comprises: determining a first biomarker index from the first temporal biomarker value; determining a second biomarker index from the second temporal biomarker value; and calculating the temporal fatness index using the first biomarker index and the second biomarker index.

In some embodiments, the computing device is configured to execute the following method steps determining if the subject is in lipolysis (fat loss) stage or lipogenesis (fat gain) stage based on the temporal fatness index; and displaying the determination on the user device. In some embodiments, determining the lipolysis (fat loss) stage when the temporal fatness index is below a first threshold value and the lipogenesis (fat gain) stage when the temporal fatness index is above a first threshold value.

In some embodiments, the method further comprises calculating a periodic fatness index by integrating the temporal fatness index over a predetermined period of time; and displaying the periodic fatness index on the user device. In some embodiments, the predetermined period is 24 hours, and the periodic fatness index is a daily fatness index.

In some embodiments, the method further comprises determining if the subject lost fat or gained fat during the predetermined period of time, based on the daily fatness index; and displaying the result on the user devise.

In some embodiments, the method further comprises receiving from the user device information related to the temporal food intake of the subject during the predetermined period of time; determining preferable food types for the subject based on the information, the temporal fatness index and the periodic fatness index; and displaying the preferable food types on the user device. In some embodiments, the first temporal biomarker value is indicative of the temporal concentration of one or more of: insulin, glucose and C-peptide in the body fluid. In some embodiments, the second temporal biomarker value is indicative of the concentration of one or more of glycerol, and free fatty acids in the body fluid.

In some embodiments, the method further comprises determining a fatness trend and wherein displaying comprises displaying the fatness trend. In some embodiments, the method further comprises providing dietary recommendations to the user.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram, depicting a system for managing the fat trend of a subject according to some embodiments of the invention;

FIG. 1B is a block diagram, depicting a computing device which may be included in a system for managing the fat trend of a subject according to some embodiments;

FIG. 2 is a flowchart of a method of managing the fat trend of a subject according to some embodiments of the invention;

FIG. 3 is a graph showing a change in the amount of glycerol and glucose in the interstitial fluid of the subject according to some embodiments of the invention;

FIGS. 4A and 4B are graphs showing colorimetric and electrochemical methods for measuring glycerol vs. direct chemical measurement of glycerol according to some embodiments of the invention;

FIG. 5 growth curves of three mice groups, each received a different diet regimen according to some embodiments of the invention;

FIGS. 6A and 6B are graphs showing measurements of blood glucose and serum glycerol at different diet regimens according to some embodiments of the inventions;

FIGS. 7A and 7B are graphs showing measurements of serum free fatty acids (FFA) and serum insulin at different diet regimens according to some embodiments of the inventions;

FIG. 8 is a graph showing the correlation between the serum glycerol and the basal metabolic rate according to some embodiments of the invention; and

FIGS. 9A and 9B are graphs showing blood glucose and serum glycerol levels during prolonged fasting at different diet regimens according to some embodiments of the inventions;

It will be appreciated that for simplicity and clarity of illustration, elements shown on the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize that the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Some aspects of the invention are directed to a system and method for combating obesity and overweight by monitoring the fat trend of a subject. For example, two sensors may be attached to a subject's belly, partially penetrating the dermis to reach a subcutaneous interstitial liquid, for measuring values indicative of the visceral fat trend (the gaining and loosing of fat). In another example, two sensors may be placed on the subject's skin for measuring values indicative of the visceral fat trend from the sweat on the subject's skin. Alternatively, the subject sweat may be collected from the skin and the two sensors may be placed inside the collected sweat. The sensors may measure a first biomarker indicative of the insulin levels (e.g., glucose) and a second biomarker indicative of fatty acids formation or consumption (e.g., glycerol). These two biomarkers may serve as potential indicators for assessing the quantity of free fatty acids and glycerol within the body fluid, as well as providing insights into their metabolic fate, including whether they undergo conversion into fat through lipogenesis or are utilized by the user through lipolysis.

As used herein, the term body fluid may refer to fluids produced by the body. For example, to subcutaneous interstitial liquid, sweat, blood, saliva, etc.

Aspects of the invention may be related to finding the “Glycerol Zero” point, which is a reference point for measuring the fatness index (e.g., obesity trend). The lipid mobilization can be divided into three states/stages. The first stage is the fed state, which commences approximately 30 minutes after a meal and continues for around 120 minutes. During this period, the body primarily relies on glucose as an energy source while storing triglycerides in adipose tissues. This process, also referred to as lipogenesis, fat storage, or fat gain, involves an transient increase in the levels of glycerol and free fatty acids (FFA) in the blood and the body fluid due to the breakdown of lipoprotein-associated triglycerides, facilitated by the enzyme LPL (lipoprotein lipase), and varies based on the fat content in the ingested food.

The second stage is the short term fasting state. This state begins approximately 120 minutes after a meal. During this phase, energy is derived from the conversion of glycogen to glucose, primarily taking place in the liver. Consequently, there is no transfer of glycerol or FFA from the digestive system to adipose tissues, nor is there a movement of glycerol and FFA from adipose tissues the blood and then to target tissues. As a result, the levels of glycerol and FFA in both blood and corresponding intercellular fluid remain minimal, therefore being referred to as the “Glycerol Zero” point and may serve as a baseline for fatness index trend analysis and the identification of dietary disorders.

The third stage is the prolonged fasting state, which typically begins around 240 minutes after a meal. After approximately 4 hours of fasting, the body begins to shift its primary source of energy from glucose to stored fat. When one consumes food, the body breaks down carbohydrates into glucose, which is the preferred fuel source for the cells. However, during prolonged fasting or prolonged periods without food intake, the body's glycogen stores become depleted, and it starts to rely on fat as an energy source through the process of lipolysis. The stored triglycerides are broken down into FFA and glycerol, which are then utilized as a source of energy. Therefore, during this phase, the levels of glycerol and FFA in the bloodstream and the corresponding body fluid (e.g., subcutaneous interstitial liquid, sweat, etc.) during are contingent upon and can serve as index for the individual's basal metabolic rate (BMR).

Some aspects of the invention may allow a user to track his/her own fat trends and align his/her meals with the circadian rhythm. The device may track all meal hours alerting deviations and sending “heads up” messages to make sure the circadian rhythm is followed. Best circadian rhythm will be calculated and will be informed to the user according to lipolysis vs. lipogenesis.

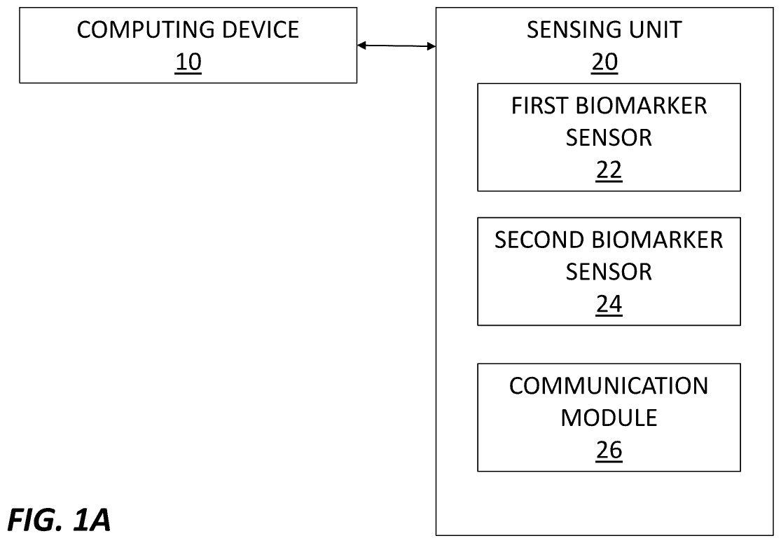

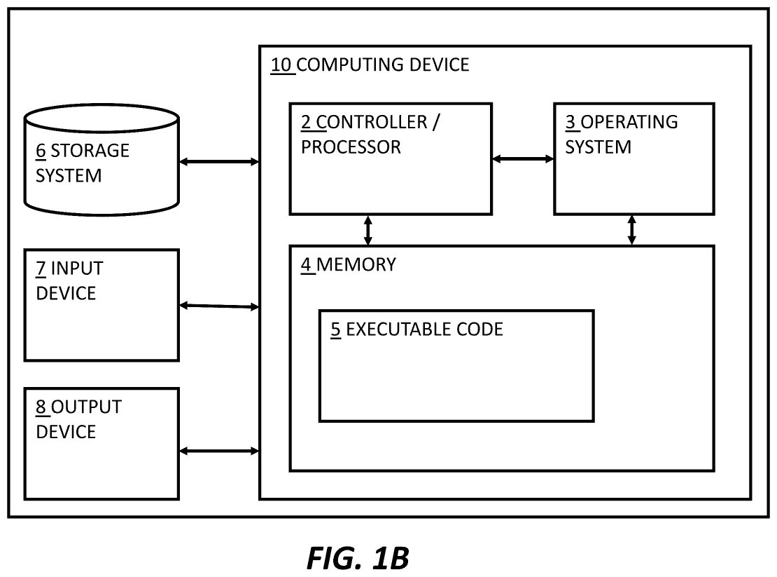

Reference is now made to FIG. 1A, which is a block diagram of a system for managing the fat trend of a subject according to some embodiments of the invention. System 100 may include a computing device, such as computing device 10 illustrated and discussed with respect to FIG. 1B or any suitable computing device or computing platform, such as a cloud-based computing platform. System 100 may further include a sensing unit 20 comprising at least one first biomarker sensor 22 and at least one second biomarker sensor 24. Sensing unit 20 may further include a communication unit 26 configured to communicate with an external computing device, such as computing device 10, or any other computing device or user device.

Sensing unit 20 may or may include a wearable unit. For example, sensing unit 20 may be embedded in a garment, or a watch, or one or more sensor of sensing unit 20 may be wearable or embedded in a wearable device. In some embodiments, sensors 22 and 24 are configured to at least partially penetrate the dermis to reach a subcutaneous interstitial fluid. In some embodiments, sensors 22 and 24 are configured to collect/measure sweat content. In some embodiments, first biomarker sensor 22 may be configured to continuously measure insulin, glucose and/or C-peptide in the body fluid. In a nonlimiting example, first biomarker sensor 22 may be a continuous glucose monitor (CGM) of any type known in the art.

In some embodiments, second biomarker sensor 24 may be any sensor configured to continuously measure one or more of glycerol, and free fatty acids (FFA) in the body fluid. A first nonlimiting example for glycerol sensor may include amperometric biosensor based on the enzyme glycerol oxidase (GO) for glycerol determination. Such an amperometric biosensor may include a transducer covered by immobilized GO being immobilized on the transducer surface. The immobilization is conducted by electrochemical polymerization of the GO preparation in polymer poly(3,4-ethylenedioxythiophene). The glycerol determination by amperometric system is based on the enzymatic reaction:

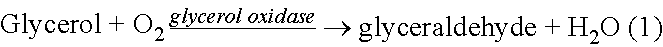

The process of glycerol enzymatic transformation results in generating electrochemically active substance and hydrogen peroxide oxidation which causes the formation of electrons measurable by means of an amperometric transducer.

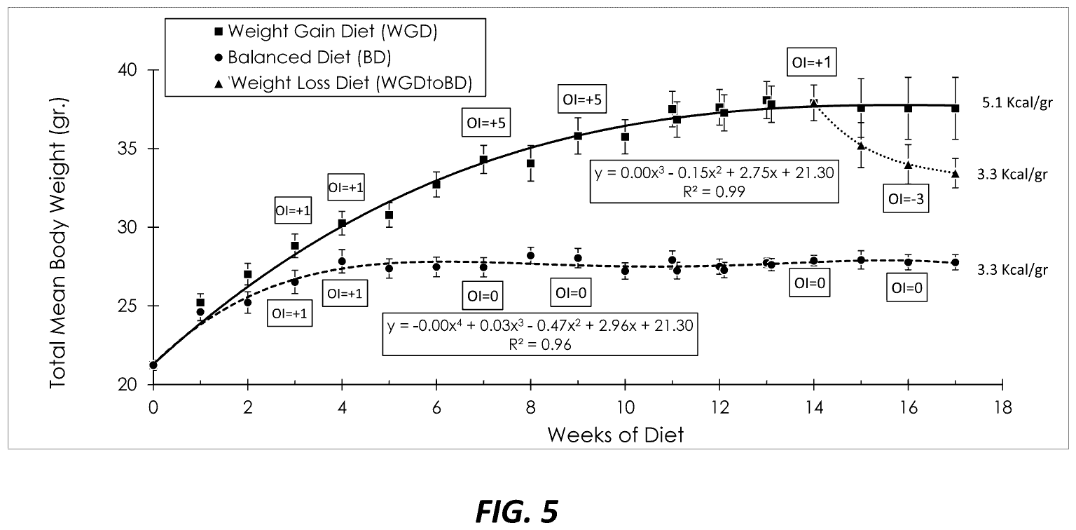

Another nonlimiting example may be the commercial in-vivo Glycerol Lite Sensing System by Zimmer and Peacock (ZP) available for purchasing on www.zimmerpeacock.com. Additional nonlimiting methods include colorimetric method and electrochemical method, discussed with respect to FIGS. 5A and 5B.

In some embodiments, communication unit 26 may be any suitable wireless of wired communication module. For example, communication unit 26 may include a Bluetooth device, a modem, a cellphone modem, and the like. In some embodiments, communication unit 26 may be configured to receive time-dependent (temporal) measurements of first temporal biomarker value from first biomarker sensor 22 and temporal measurements of second temporal biomarker value from second biomarker sensor 24 and to send the first temporal biomarker value and the second temporal biomarker value to computing device 10 for further processing.

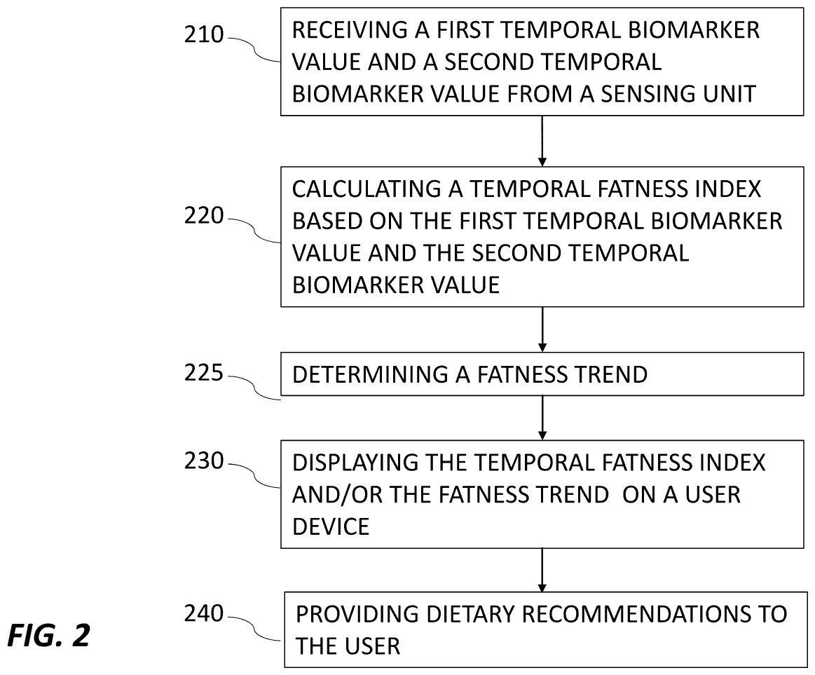

Reference is now made to FIG. 1B, which is a block diagram depicting a computing device, which may be included in a system for managing fat trend of a subject, according to some embodiments. Computing device 10, may be an external device or may be a computing unit attached or wirelessly connected to sensing unit 20. In some embodiments, computing device 10 may be configured to calculate a temporal fatness index based on the first temporal biomarker value and the second temporal biomarker value and to display the temporal fatness index on a user device, as discussed herein below with respect to the flowchart of FIG. 2.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein, for example, method of managing fat trend of a subject.

Executable code 5 may be any executable code, e.g., an application, a program, a process, task or script. Executable code 5 may be executed by processor or controller 2 possibly under control of operating system 3. For example, executable code 5 may be an application that may manage fat trend of a subject as further described herein. Although, for the sake of clarity, a single item of executable code 5 is shown in FIG. 2, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data related to biomarkers values of a subject or a group of subjects may be in storage system 6 and may be loaded from storage system 6 into memory 4 where it may be processed by processor or controller 2. In some embodiments, some of the components shown in FIG. 1B may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. 2 which is a method of managing fat trend of a subject according to some embodiments of the invention. The method of FIG. 2 may be conducted by computing device 10 or by any other suitable computing device.

In step 210, a first temporal biomarker value and a second temporal biomarker value in the body fluid of the subject may be received from a sensing unit comprising a first biomarker sensor and a second biomarker sensor. For example, first temporal biomarker value may be received from first biomarker sensor 22 and second temporal biomarker value may be received from second biomarker sensor 24. In some embodiments, the first temporal biomarker value is indicative of the temporal concentration of one or more of: Insulin, Glucose and C-peptide in the body fluid. In some embodiments, the second temporal biomarker value is indicative of the concentration of one or more of Glycerol, and free fatty acids in the body fluid.

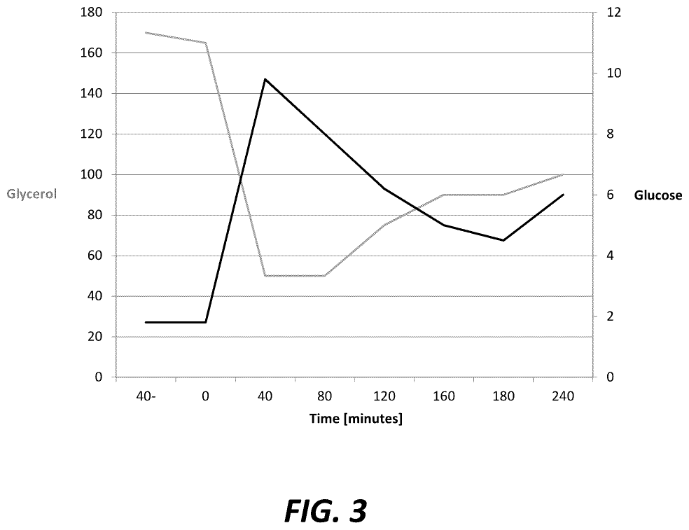

In a nonlimiting example, the first temporal biomarker value may be temporal glucose levels and the second temporal biomarker value may be temporal glycerol levels. An example for measurement of temporal glucose levels and temporal glycerol levels is given in FIG. 3 measured from the subcutaneous interstitial fluid. The temporal glucose levels (measured in μM) and glycerol levels (measured in μ-mol) were measured for 280 minutes, 40 minutes before the subject ate a meal (meal time at t=0), and 240 minutes after the meal. As one can see, there is a sharp rise in the level of glucose right after the meal (about 30 min. after the meal) followed by a slow decrease and a moderate rise in glycerol level over a long period of time (more than 240 min.).

In some embodiments, the method may further include determining the “Glycerol Zero” point for all biomarkers and may serve as a baseline for each biomarker. The “Glycerol Zero” point may be determined from biomarker values received between 120 to 240 minutes after the meal, during the short-term fasting state.

In step 220, a temporal fatness index may be calculated, based on the first temporal biomarker value and the second temporal biomarker value. In some embodiments, the temporal fatness index may be calculated by determining a first biomarker index from the first temporal biomarker value, determining a second biomarker index from the second temporal biomarker value and calculating the temporal fatness index using the first biomarker index and the second biomarker index. Looking at the nonlimiting example of FIG. 3, the glucose index may be determined as (−1) for all glucose levels lower than 3 and (+1) for all glucose levels equal to or higher than 3. The glycerol index may include the glycerol level multiplied by a constant. The temporal fatness index may be determined by multiplying the glycerol index by the glucose index.

In step 225, a fatness trend may be determined. In some embodiments, the method may further include determining whether the subject is in either a lipolysis (fat loss) stage or a lipogenesis (fat gain) stage, based on the temporal fatness index. In some embodiments, the method may further include determining ae lipolysis (fat loss) stage when the temporal fatness index is below a first threshold value, and a lipogenesis (fat gain) stage when the temporal fatness index is above a first threshold value. For example, if the temporal fatness index is a positive value the subject is in a lipogenesis (fat gain) stage, and if the temporal fatness index is a negative value, the subject is in a lipolysis (fat loss) stage. The higher the absolute value of the temporal fatness index the faster are either the fat loss (negative values) or fat gain (positive values).

In some embodiments, determining the trend may include comparing each temporal biomarker index to the “Glycerol Zero” point of that biomarker, using an appropriate algorithm and known cutoff values.

In step 230. The temporal fatness index is displayed on a user device, for example, a user device (e.g., smartphone, smartwatch, laptop, table, etc.) associated with the subject, a caregiver, a dietician, a doctor and the like. For example, a positive fatness index may be displayed in a red font or as a red marker indicating lipogenesis. In yet another example, a negative fatness index may be displayed in a green font or as a green marker indicating lipolysis.

In some embodiments, the method may include calculating a periodic fatness index by integrating the temporal fatness index over a predetermined period of time (e.g., 12 hours, 24 hours, one week, etc.) and displaying the periodic fatness index on the user's device. For example, a daily fatness index may be calculated by integrating the temporal fatness index over 24 hours. If the daily fatness index is a positive value, then the subject gained fat during that day; and if the daily fatness index is a negative value, then the subject lost fat during that day.

In step 240, dietary recommendations may be provided to the user. In some embodiments, the calculated periodic fatness index may be used to determine preferable food types that encourage a desired fat trend (e.g., fat loss or fat gain), specifically for the subject. In some embodiments, the method may include receiving from the user device information related to the temporal food intake of the subject during the predetermined period of time. The user may enter his daily meals using his personal user device, for example, by selecting from a list of food items. The user may also mark which food item was eaten at a specific meal or specific time. Accordingly, computing device 10 may determine which food type, or group of food types encourage fat lose and which causes fat gain. The preferable food types may be presented to the subject on the user's device.

In some embodiments, the method may include correlating the temporal fatness index with the circadian rhythm of the subject, to optimize the meal hours.

### Experimental Results

### Glycerol Measurements

While glucose measurements are now widely conducted using simple domestic devices (e.g., first biomarker sensor 22), by nonprofessional users, simple glycerol measurement devices/sensors are still scarcely used. There are several sensing methods that can be good candidates for a simple measurement of serum glycerol (e.g., second biomarker sensor 24). Experiments showing the validity of indirect glycerol measurements were done for two candidates, colorimetric method using Glycerol Colorimetric Assay Kit (Sigma-Aldrich, cat #MAK117), and electrochemical method using Discrete Glycerol Biosensor, by Zimmer-Peacock.

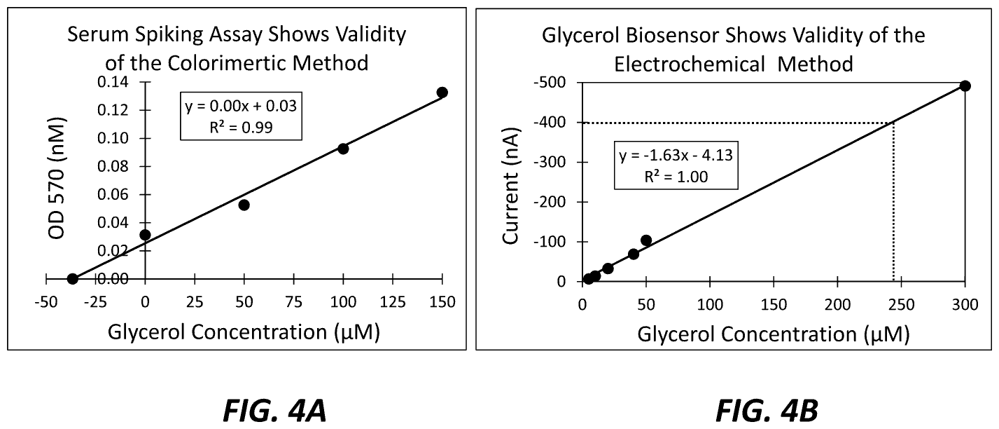

Reference is now made to FIGS. 4A and 4B which are graphs showing measurements of values indicative of the concentration of serum glycerol using two indirect measurements. FIG. 4A shows measurements of the optical density (absorbance) at a wavelength of 570 nm (OD 570 received from standard solutions of a known glycerol concentration (in μM) using the Glycerol Colorimetric Assay kit. As shown in FIG. 4A there is an excellent match (R²=0.99) between the optical density (absorbance) and the glycerol levels.

FIG. 4B shows measurements of the current (in nA units) received using the Discrete glycerol biosensor in comparison to the direct chemical measurements. As shown in FIG. 4B there is an excellent match (R²=1) between the two sets of measurements.

Accordingly, both methods can be used for measurements of values indicative of the amount of glycerol in the serum, as well as in the interstitial fluid of the subject.

### Metabolic Study in Mice

Reference is now made to FIG. 5, which shows growth curves of the groups of C57BL/6J male mice according to some embodiments of the invention. Two groups of male mice were kept on either balanced diet (BD-TD.120455 from TEKLAD, 22%/61%/17% kcal from protein/carbohydrates/fat; total 3.3 kcal/gr) or weight gain diet (WGD-TD.0614 from TEKLAD, 18%/21%/60% kcal from protein/carbohydrates/fat; total 5.1 kcal/gr) for up to 18 weeks, and monitored weekly for weight and general health. A third group was kept for 13 weeks on WDG, and then switched to BD, to cause their weight loss, for an additional 4 weeks. Each group included 5 mice. At the morning of each assay, after 15 hrs overnight fast, mice were refed with either of the diets, blood samples were collected at fasting and at fed (1-hour postprandial) states. Blood glucose levels (measured using an Accu-Chek glucometer) and the serum glycerol (measured using the gold standard chemical testing by Glycerol Colorimetric Assay Kit, Sigma-Aldrich, cat #MAK117). Were then used to calculate the Obesity Index (OI-boxed numbers) according to Obesity Trend Predictability algorithm (OBCTor's).

The growth curves of the groups of mice could be accurately modeled using 4th-order polynomial equations. Analyzing the equations shows that while the curve does not strictly adhere to the mathematical definition with a precisely zero derivative, it is apparent from the 12th week onwards that it aspires to reach a plateau. This aspiration is observed as the increases in value become infinitesimally small. Accordingly, a paired t-test conducted on the average weekly group weights revealed that, from the 11th week onward of the HFD, differences in mean weight gains in both groups were statistically insignificant.

The corresponding data is given in table 1, which proves the suitability of insulin and glucose as biomarkers of diet regimen. The scores given to each biomarker level are F (fasting) and P (Postprandial) and were calculated using an appropriate algorithm and known cutoff values.

TABLE 1
Suitability of insulin and Glucose as Biomarkers of Diet Regimen

| feed | Diet Regimen | Insulin | | Er- | Glucose | | Er- |
|---|---|---|---|---|---|---|---|
| state | (Long/short) | (ng/ml) | Score | ror | (mg/dl) | Score | ror |
| Fasting | CHOW/chow | 1.2 | F | 5 of | 34 | F | 2 0f |
| | | 0.5 | F | 16 | 30 | F | 16 |
| | | 9.7 | P | | 40 | F | |
| | | 1.2 | F | | 64 | F | |
| | | 9.7 | P | | 72 | F | |
| | | 1.8 | F | | 51 | F | |
| | HFD/chow | 2.3 | F | | 85 | F | |
| | | 4.2 | P | | 96 | F | |
| | HFD/hfd | 1.0 | F | | 68 | F | |
| | | 2.1 | F | | 93 | F | |
| | | 4.1 | P | | 81 | F | |
| | | 9.8 | P | | 77 | F | |
| | | 3.2 | F | | 108 | P | |
| | | 3.2 | F | | 136 | P | |
| | HFDtoCHOW | 2.6 | F | | 58 | F | |
| | | 1.9 | F | | 64 | F | |
| Post- | CHOW/chow | 5.3 | P | 5 of | 117 | P | 6 of |
| prandial | | 4.5 | P | 22 | 118 | P | 16 |
| | | 4.4 | P | | 118 | P | |
| | | 4.3 | P | | 86 | P | |
| | | 2.6 | F | | 86 | P | |
| | | 2.5 | F | | 88 | P | |
| | | 5.5 | P | | 117 | P | |
| | | 3.5 | P | | 104 | P | |
| | HFD/chow | 3.4 | P | | 103 | P | |
| | | 4.2 | P | | 76 | F | |
| | | 7.1 | P | | 94 | F | |
| | | 8.6 | P | | 111 | P | |
| | | 4.6 | P | | 94 | F | |
| | | 2.3 | F | | 88 | F | |
| | | 5.0 | P | | 95 | F | |
| | HFD/hfd | 5.6 | P | | 117 | P | |
| | | 2.5 | F | | 122 | P | |
| | | 4.7 | P | | 119 | P | |
| | | 8.9 | P | | 115 | P | |
| | | 5.4 | P | | 88 | F | |
| | | 6.8 | P | | 128 | P | |
| | | 13.0 | P | | 133 | P | |
| | HFDtoCHOW | 4.4 | P | | 111 | P | |
| | | 2.8 | F | | 111 | P | |
| | | 6.7 | P | | 102 | P | |

Table 1 clearly shows that serum insulin and blood glucose can be served as biomarkers for identifying the fasting and the postprandial stages.

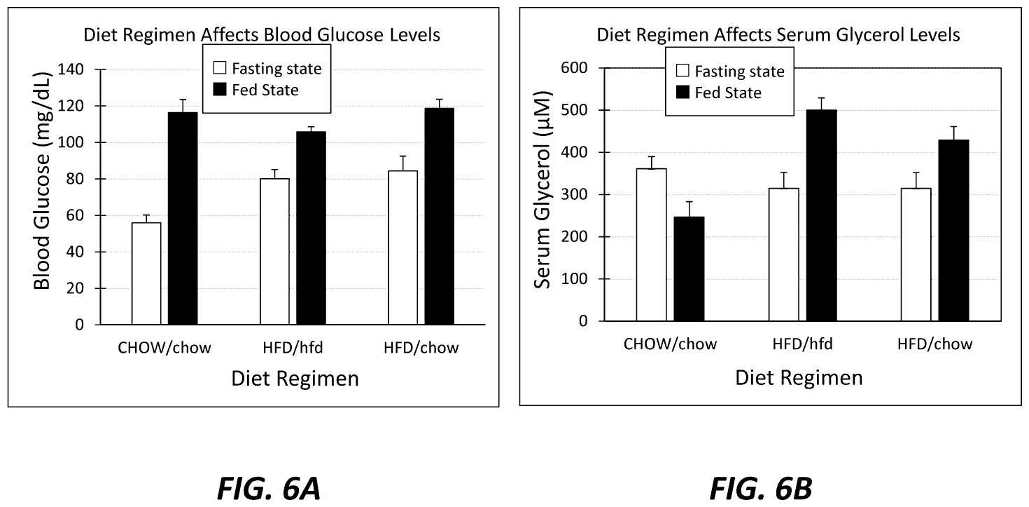

Reference is now made to FIGS. 6A and 6B which include graphs showing measurements of blood glucose and serum glycerol at different diet regimens according to some embodiments of the inventions. The blood glucose (e.g., the first temporal biomarker) was measured using an Accu-Chek glucometer, and the serum glycerol (e.g., second temporal biomarker) was measured using the gold standard chemical testing by Glycerol Colorimetric Assay Kit (Sigma-Aldrich, cat #MAK117).

The mice were kept on either balanced diet (BD, CHOW) or weight gain diet (WGD, HFD) for up to 18 weeks, and assayed weekly for biomarker blood levels. At the morning of each assay, after an overnight fast, the mice were refed with a short-term diet (for 1 hr.) of either chow (CHOW/chow), HFD (HFD/hfd), or chow (HFD/chow), for the whole period of blood sampling (high case and low case letters represent long-term and subsequent short-term diet regimens, respectively). Blood samples were collected at the fasting and at the fed (60 min postprandial) states, and used for the determination of blood glucose and serum glycerol levels.

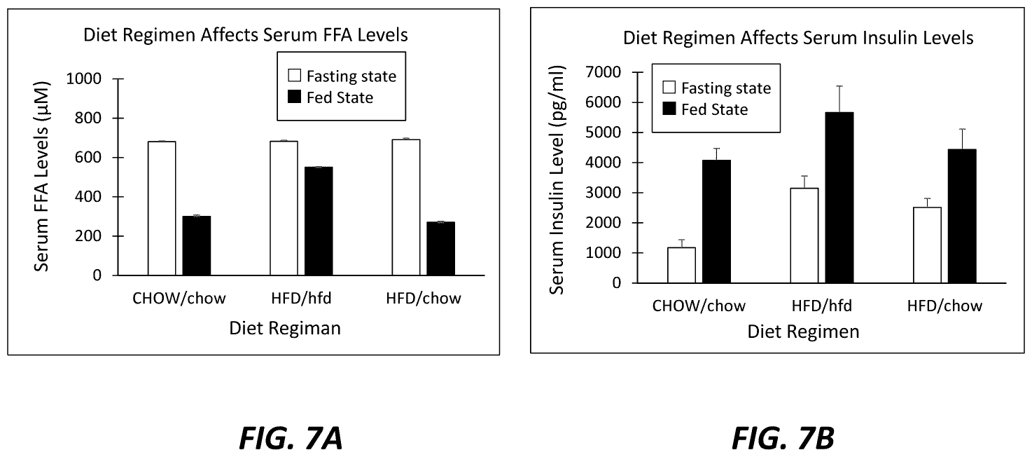

A similar approach was made with different biomarkers, serum FFA and serum insulin. Reference is now made to FIGS. 7A and 7B which are graphs showing measurements of serum FFA (e.g., the first temporal biomarker) and serum insulin (e.g., the second temporal biomarker) at different diet regimens according to some embodiments of the inventions. The serum FFA and serum insulin biomarkers were measured using appropriate ELISA kits, at the same times and conditions as the glucose and glycerol.

It is clear from the results shown in FIGS. 6A, 6B, 7A and 7B that glucose and serum glycerol, serum FFA and serum insulin are all very good candidates for temporal biomarkers according to embodiments of the invention. All the measured biomarkers can be used for calculating the temporal fatness index, and therefore can be used for identifying lipolysis stage or lipogenesis stage.

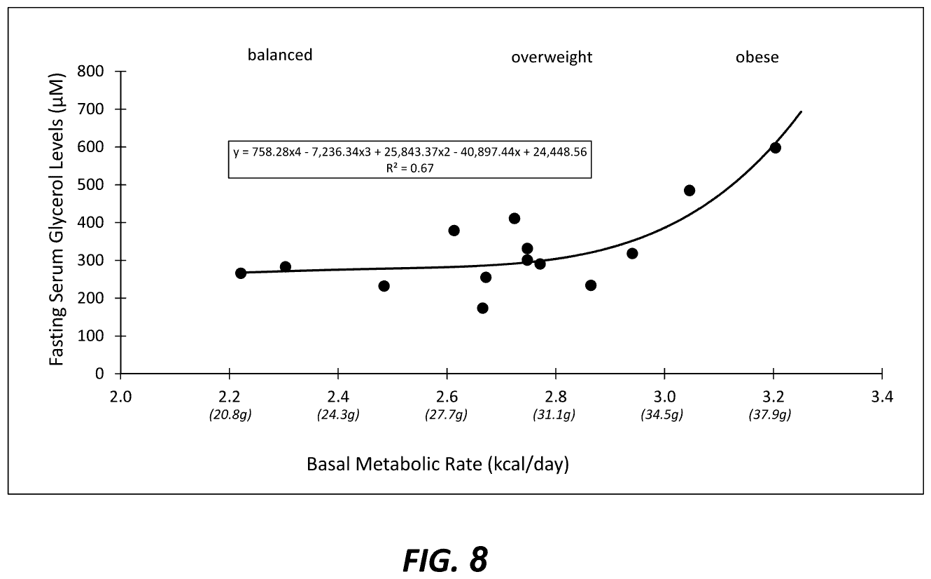

Reference is now made to FIG. 8 which is a graph showing the correlation between the serum glycerol and the basal metabolic rate according to some embodiments of the invention. The tests were conducted on a group of C57BL/6J male mice. The mice were kept on either normal chow (CHOW) or 60% high fat diet (HFD) for up to 18 weeks, and assayed weekly for total body weight (TWB) and biomarker blood levels. BMR was calculated according to the formula: BMR (kcal/day)=0.0585×TBW+0.981, which is a specific adaptation to male C57BL mice of the Harris-Benedict equation. Serum glycerol levels were measured in blood samples collected after 15-hrs overnight fast, and plotted against the calculated BMR (the numbers in parenthesis represent the corresponding TBW). The plot could be accurately modeled using 4th-order polynomial equation as shown on the graph, with significant positive correlation between BMR and fasting serum glycerol levels. This data clearly suggests that fasting glycerol levels at rest can be used as indicator of BMR.

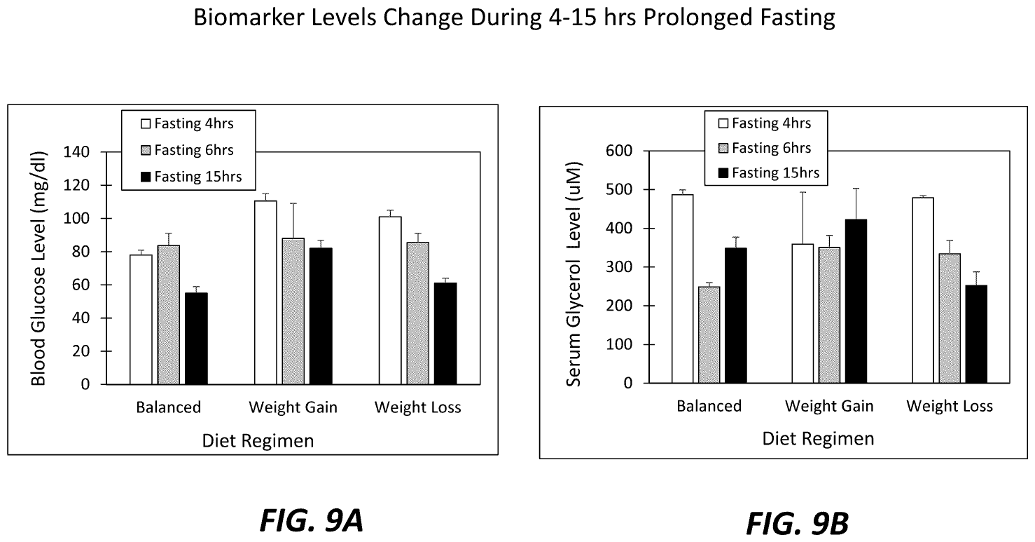

Reference is now made to FIGS. 9A and 9B which are graphs showing blood glucose and serum glycerol levels during various periods of fasting, and at different diet regimens according to some embodiments of the inventions. The graphs follow the changes in blood glucose (FIG. 9A) and serum glycerol (FIG. 9B) during 4, 6 and 15 hrs. of fasting. As expected, the blood glucose levels were generally declining over time, except for the balanced diet that shows a minor increase in blood glucose levels between 4 to 6 hours. During the prolonged fasting stage, the fat lipolysis process takes place, which involves the conversion of triglycerides into glycerol and FFA. Therefore, for both the balanced diet and the weight loss diet, the higher levels of serum glycerol were measured already after 4 hours of fasting (e.g., 4 hours from the end of the meal). The serum glycerol levels measured in the mice that were fed with a weight gain diet, showed a reverse trend, namely, a increase in serum glycerol level was observed after fasting for 15 hours. As shown, for losing weight, one must have a decrease in both blood glucose levels and serum glycerol levels during the prolonged fasting stage.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for managing fat trend of a subject, the system comprising:
a sensing unit; and
a computing device
wherein the sensing unit comprises:
a first sensor configured to measure a first temporal biomarker value in a body fluid of the subject;
a second sensor configured to measure a second temporal biomarker value in the body fluid of the subject;
a communication module configured to transmit the measured temporal biomarkers values to the computing device, and
wherein the computing device is configured to:
a. receive the first temporal biomarker value and the second temporal biomarker value from the sensing unit;
b. calculate a temporal fatness index based on the first temporal biomarker value and the second temporal biomarker value; and
c. display the temporal fatness index on a user device.

2. The system of claim 1, wherein calculating the temporal fatness index comprises:
a. determining a first biomarker index from the first temporal biomarker value;
b. determining a second biomarker index from the second temporal biomarker value;
c. calculating the temporal fatness index using the first biomarker index and the second biomarker index.

3. The system of claim 1, wherein the computing device is configured to:
a. determine if the subject is in Lipolysis (fat loss) stage or Lipogenesis (fat gain) stage based on the temporal fatness index; and
b. display the determination on the user device.

4. The system of claim 3, wherein determining the Lipolysis (fat loss) stage when the temporal fatness index is below a first threshold value and the Lipogenesis (fat gain) stage when the temporal fatness index is above a first threshold value.

5. The system of claim 1, wherein the computing device is configured to:
a. calculate a periodic fatness index by integrating the temporal fatness index over a predetermined period of time; and
b. display the periodic fatness index on a user device.

6. The system of claim 5, wherein the predetermined period is 24 hours, and the periodic fatness index is a daily fatness index.

7. The system of claim 5, wherein the computing device is configured to:
a. determine if the subject lost fat or gained fat during the predetermined period of time, based on the daily fatness index; and
b. display the result on the user devise.

8. The system of claim 5, wherein the computing device is configured to:
a. receive from the user device information related to the temporal food intake of the subject during the predetermined period of time;
b. determine preferable food types for the subject based on the information, the temporal fatness index and the periodic fatness index;
c. display the preferable food types on the user device.

9. The system of claim 1, wherein the at least two sensors are configured to at least partially penetrate the dermis to reach a subcutaneous body fluid.

10. The system of claim 1, wherein the first temporal biomarker value is indicative of the temporal concentration of one or more of: Insulin, Glucose and C-peptide in the body fluid.

11. The system of claim 1, wherein the second temporal biomarker value is indicative of the concentration of one or more of Glycerol, and free fatty acids in the body fluid.

12. The system of claim 1, wherein the computing device is at least one of: a smartphone, a laptop computer, a desktop computer, a smartwatch, a tablet computer, and a wearable computing device.

13. A method of managing fat trend of a subject, comprising:
a. receiving a first temporal biomarker value and the second temporal biomarker value in a body fluid of the subject, from a sensing unit comprising a first biomarker sensor and a second biomarker sensor;
b. calculating a temporal fatness index based on the first temporal biomarker value and the second temporal biomarker value; and
c. displaying the temporal fatness index on a user device.

14. The method of claim 13, wherein calculating the temporal fatness index comprises:
a. determining a first biomarker index from the first temporal biomarker value;
b. determining a second biomarker index from the second temporal biomarker value;
c. calculating the temporal fatness index using the first biomarker index and the second biomarker index.

15. The method of claim 13, further comprising:
a. determining if the subject is in Lipolysis (fat loss) stage or Lipogenesis (fat gain) stage based on the temporal fatness index; and
b. displaying the determination on the user device.

16. The method of claim 15, wherein determining the Lipolysis (fat loss) stage when the temporal fatness index is below a first threshold value and the Lipogenesis (fat gain) stage when the temporal fatness index is above a first threshold value.

17. The method of claim 13, further comprising:
a. calculating a periodic fatness index by integrating the temporal fatness index over a predetermined period of time; and
b. displaying the periodic fatness index on the user device.

18. The method of claim 17, wherein the predetermined period is 24 hours, and the periodic fatness index is a daily fatness index.

19. The method of claim 17, further comprising:
a. determining if the subject lost fat or gained fat during the predetermined period of time, based on the daily fatness index; and
b. displaying the result on the user devise.

20. The method of claim 17, further comprising:
a. receiving from the user device information related to the temporal food intake of the subject during the predetermined period of time;
b. determining preferable food types for the subject based on the information, the temporal fatness index and the periodic fatness index; and
c. displaying the preferable food types on the user device.
